(19) United States
(12) Patent Application Publication
(10) Pub. No.: US 2017/0020593 A1
(43) Pub. Date: Jan. 26, 2017

(54) DEVICE FOR GENERATING VAPOUR FOR INJECTION INTO A HUMAN OR ANIMAL VESSEL

(71) Applicant: MIRAVAS, Besançon (FR)

(72) Inventor: Nicolas Rauber, Besançon (FR)

(21) Appl. No.: 15/124,504

(22) PCT Filed: Mar. 10, 2015
(86) PCT No.: PCT/FR2015/050587

§ 371 Date: Sep. 8, 2016

(30) Foreign Application Priority Data
Mar. 11, 2014 (FR) FR1400584

(51) Int. Cl. A61B 18/04; A61M 5/44; A61M 39/24; A61M 5/142

(52) U.S. Cl. CPC A61B 18/04; A61M 5/142; A61M 5/44; A61M 39/24; A61M 2205/36; A61M 2205/362; A61B 2018/00404

(57) ABSTRACT

A handpiece for injecting steam into a human or animal vessel in order to heat the vessel, including a steam generator for generating steam from a liquid and a connection member for connecting a catheter or a needle that is to inject the steam into the vessel. The steam generator includes a reservoir to contain the liquid, the reservoir being equipped with electrical heating means for heating the liquid in order to vaporize it and produce a stream of steam that diffuses via the connection member. The steam generator also includes a first nonreturn outlet valve interposed between the reservoir and the connection member for preventing fluid from passing from the connection member toward the reservoir. The first irst nonreturn outlet valve being a pressure-regulating valve which opens to allow steam to pass from the reservoir toward the connector when the pressure in the reservoir has reached a threshold value.

### RELATED APPLICATIONS

This is the national stage, under 35 USC 371, of PCT application PCT/FR2015/050587, filed on Mar. 10, 2015, which claims the benefit of the Mar. 11, 2014 priority date of French Application 1400584, the content of which is herein incorporated by reference.

### FIELD OF INVENTION

The invention relates to a device for the thermal treatment of venous conditions by the thermally regulated injection of steam.

### BACKGROUND OF THE INVENTION

More specifically, the invention relates to a device for injecting steam generated from a liquid such as water or physiological liquid into a human or animal vessel in order to heat it, typically with a view to it closing itself by contracting. In concrete terms, when the steam reaches the inside of the vessel it condenses and therefore gives up its sensible heat and its latent heat of condensation, making it possible to effectively heat the vessel in question.

This type of treatment is carried out for example in order to treat varicose veins, hemorrhoids, warts or the like: the practitioner then uses a catheter, or indeed a needle, that he connects to a handpiece. This handpiece is provided with heating means and is connected to a cold water injection unit which can be likened to a pump and which provides pulsed small quantities of liquid with a volume of the order of twenty-four micro liters.

In operation, the catheter is introduced into the vessel that is to be treated, and the injection unit, at regular intervals, introduces pulses of cold liquid into the handpiece which comprises heating means. Each volume of liquid is vaporized by the heating means and is thus diffused into the vessel via the catheter which is connected in fluidtight manner to this handpiece.

Such apparatus, which is described in patent document FR2915872 comprises a handpiece capable of heating each pulse of water that it receives from the injection unit fairly rapidly.

In practice, such apparatus is necessarily sophisticated which means that it can be available only in a hospital environment, such a system being too expensive for a practitioner to be able to acquire it in order to carry out this type of treatment within his own medical practice.

### SUMMARY OF THE INVENTION

The object of the invention is to propose a solution for overcoming this disadvantage by proposing steam injection apparatus of low cost.

To this end, the subject of the invention is a handpiece for injecting steam into a human or animal vessel in order to heat this vessel, this handpiece comprising means for generating steam from a liquid such as water or physiological liquid and a connection member for connecting to a catheter or to a needle that is to be introduced into the vessel in order to inject the steam into same, characterized in that the steam-generating means comprise:
a reservoir intended to contain the liquid, this reservoir being equipped with electrical heating means for heating the liquid in order to vaporize it and produce a stream of steam that diffuses via the connection member, and
a nonreturn outlet valve interposed between the reservoir and the connection member for preventing fluid from passing from the connection member toward the reservoir, said outlet valve being a pressure-regulating valve which opens to allow steam to pass from the reservoir toward the connector when the pressure in the reservoir has reached a threshold value.

By virtue of this handpiece, the steam is produced without an injection unit: all of the liquid is present directly in the reservoir and is heated in its entirety in the handpiece. Because there is no longer any need for a pump, the apparatus is less expensive and is therefore accessible to a medical practice. The stream of steam may diffuse either continuously or discontinuously depending on how the outlet valve is embodied and on how the electrical heating means are embodied, as will be better seen later on.

The heating means are advantageously situated inside the reservoir so as to be surrounded by the liquid and/or the steam that is to be heated, this making it possible to minimize heat losses: the heat produced by Joule effect directly irradiates the mixture of liquid and steam that the reservoir contains. In one example, the heating means comprise an electrical resistance.

Advantageously, the reservoir is at least partially pre-filled with liquid that is to be vaporized. Thus, there is no need to add liquid during the course of a treatment.

The handpiece according to the invention may also comprise means for filling the reservoir including a nonreturn valve ensuring that liquid and/or steam cannot leave the reservoir via the filling means, particularly during use of the handpiece.

The handpiece may further comprise a purge valve for purging the reservoir; that allows air contained in the reservoir to be discharged prior to heating.

According to one embodiment of the invention, the outlet valve is a single-threshold pressure-regulating valve; thus, in the steady state, the handpiece may produce a continuous stream of steam, as will be better seen later on.

According to another embodiment suited to the production of a pulsed stream of steam, the outlet valve is a two-threshold pressure-regulating valve:
the outlet valve opening to allow the steam to pass when the pressure in the reservoir reaches a high threshold, and
the outlet valve closing to prevent the steam from passing when the pressure in the reservoir drops below a low threshold lower than the high threshold.

The invention also relates to a system comprising a handpiece as defined hereinabove and a control unit intended to be connected to this handpiece to supply heating means with electricity, this control unit comprising means for limiting the power of the electrical supply to the heating means.

In such a system, the handpiece may further comprise a thermal probe situated inside the reservoir, and the control unit may comprise servo control means for controlling the power of the electrical supply to the heating means on the basis of the temperature delivered by the thermal probe and/or on the basis of the temperature of the heating means so as to servo control the electrical power supplied to the heating means to a maximum permissible temperature in the reservoir.

The system may also comprise a catheter provided with a valve that prevents fluid from passing from the catheter toward the reservoir, for example a valve of the nonreturn type and/or a valve that opens when the pressure in the reservoir reaches a threshold value.

### BRIEF DESCRIPTION OF THE FIGURES

The invention will be better understood, and further features and advantages of the invention will become apparent in the light of the description which follows of some examples of handpieces and of systems according to the invention. These examples are given by way of nonlimiting illustration. The description should be read in conjunction with the attached drawings in which:

FIG. 1 is a schematic depiction of a steam injection system according to the invention.

FIG. 2 is a schematic depiction of a handpiece according to the invention.

### DETAILED DESCRIPTION

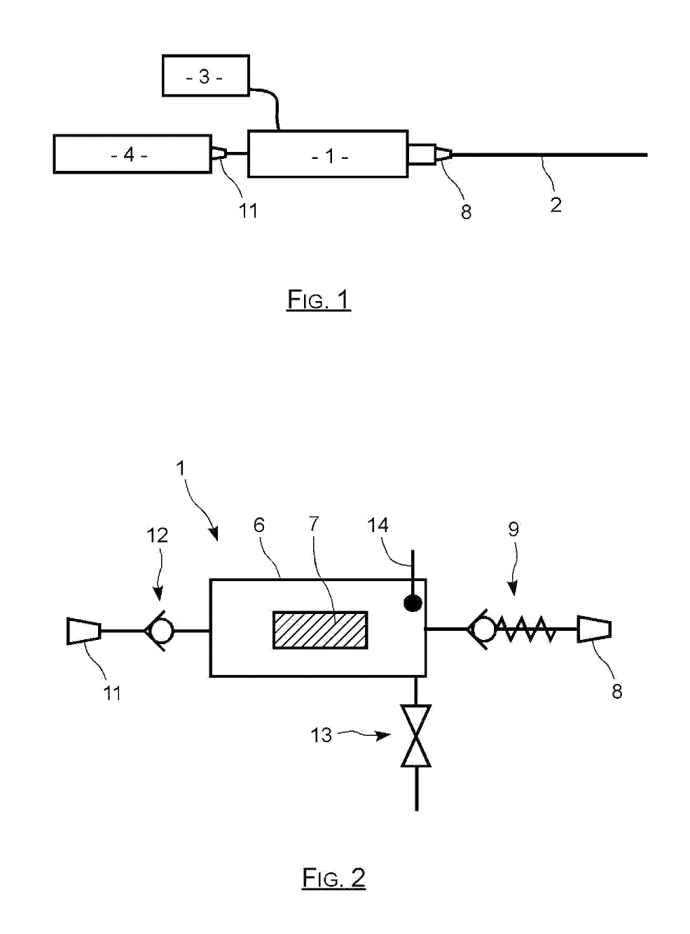

The system according to the invention which is depicted schematically in FIG. 1 comprises a handpiece 1 for generating steam by heating a liquid, so as to diffuse this steam via a catheter 2 connected to this handpiece, and a control unit 3 supplied by an electrical network and which is itself electrically connected to the handpiece 1 in order to supply it with electricity. In an alternative form (not depicted), the control unit 3 is powered by an accumulator cell or a battery of accumulator cells.

The handpiece 1 which is depicted schematically in the figures as a rectangle does in fact have an ergonomic shape that allows it to be grasped and handled easily by a practitioner holding it in his hand.

In the configuration of FIG. 1, a container 4 is connected to the handpiece 1 to fill it with a liquid such as sterile water or alternatively a physiological serum that is to be heated in order to produce the pressurized steam that is to be injected. As visible in greater detail in FIG. 2, the handpiece 1 comprises a heating reservoir 6, comprising a casing made for example of rigid plastic, containing an electrical heating element 7 of the electrical resistance type that allows the contents of the reservoir to be heated through a Joule effect.

The heating element 7 is situated inside the reservoir 6 that it equips, for example in the central region thereof, so that the external surface of this element 7 irradiates the heat produced by Joule effect toward the contents of the container, namely the liquid and/or steam. Heat losses are therefore minimized because all of the heat derived from the element 7 primarily heats the contents of the reservoir, so as to generate steam rapidly.

This heating element may have a helicoidal, straight, tubular or some other shape, and may be manufactured from various materials such as stainless steel, Inconel®, or any other suitable material. As an order of scale, for the envisioned application, its electrical resistance is comprised between one tenth and four ohms, and is, for example, one ohm.

The handpiece 1 is equipped with a dedicated connector, identified as 8, of standardized type, intended to accept a catheter 2 for connecting this catheter in a fluidtight manner to the reservoir 1 in which the steam is produced. This standardized connector is thus able to accept any catheter commonly available in medical circles. This connector is connected by a tube, not depicted, to an internal region of the reservoir so that it can pick up steam rather than liquid there: the fluid arriving at the connector 8 is thus, for example, collected in the upper part of the reservoir 6, or in the region of its connection to the reservoir.

Using the catheter connector, the practitioner selects the catheter suited to the treatment that he is to perform before connecting it to the handpiece 1 in order to proceed with this treatment. The catheter used is intended to withstand a high temperature, for example one hundred and fifty degrees, and has dimensions suited to being introduced into the vessel that is to be treated.

As depicted schematically in the example of FIG. 2, the handpiece 1 also incorporates an outlet valve 9 which is interposed between the internal casing that the reservoir 6 delimits and the connector 8 that accepts the catheter. This outlet valve 9 is a pressure-regulating valve: it opens when the pressure in the reservoir 6 reaches a certain value in order to release the steam present in the reservoir 6 into the catheter. This outlet valve 9 remains closed as long as the pressure in the reservoir 6 is insufficient. Thus, blood cannot reflux from the treated vessel toward the reservoir of the handpiece in which the steam is generated.

In the example depicted schematically in FIG. 2, this outlet valve 9 has an overall structure corresponding to that of a ball-type nonreturn valve equipped with a spring which is set at a certain value. This valve 9 thus opens when the difference in pressure between its inlet and its outlet is higher than a threshold value corresponding to the value for which the spring is set, and otherwise remains closed. When the heating means are activated, the liquid is heated, thereby increasing the pressure in the reservoir. When the pressure reaches a threshold value, the valve opens, the steam escapes from the reservoir. In a stable steady state, if the liquid is continuously heated, the steam flow rate from the valve auto adjusts in order to keep the pressure in the reservoir at the threshold value. When heating is shut off, the valve closes, a depression is created inside the reservoir, and this depression causes the reservoir to fill by drawing in liquid contained in the container 4.

In practice, this outlet valve 9 thus makes it possible to regulate the pressure in the reservoir 6 to a predetermined value that in fact corresponds to a value for the temperature of the steam generated in the reservoir. For example, in the case of water, steam at one hundred and ten degrees corresponds to a pressure of fourteen tenths of a bar in the reservoir; steam at one hundred and twenty degrees corresponds to a pressure of two bar in the reservoir; and steam at one hundred and fifty degrees corresponds to a pressure of five bar in the reservoir.

In other words, by limiting the pressure in the reservoir 6, the outlet valve 9 also allows the temperature in the reservoir to be controlled, given that in operation, the pressure in the reservoir 6 is the saturation vapor pressure of the mixture of liquid and steam. The setting of the outlet valve 9 thus directly governs the temperature of the steam produced.

It will be noted that if this setting is close to zero, the pressure stabilizes at one bar in the reservoir 6, which gives rise to the production of steam at one hundred degrees leaving the valve 9. The valve 9 then acts solely as a nonreturn valve.

This valve 9 is dimensioned and its calibration set at the time of its manufacture, for example to open when the pressure in the reservoir 6 reaches two bar, so as to release steam at a temperature close to one hundred and twenty degrees.

In an alternative form (not depicted), the outlet valve 9 is a two-threshold pressure-regulating valve:
the outlet valve opens to allow the steam to pass when the pressure in the reservoir reaches a high threshold, and
the outlet valve closes to prevent the steam from passing when the pressure in the reservoir drops below a low threshold lower than the high threshold.

Thus, in the steady state, the valve remains closed cyclically for the time needed for the pressure in the reservoir to progress from the low threshold (corresponding to valve closure) to the high threshold (corresponding to valve opening). The handpiece thus produces a pulsed stream of steam. The quantity of steam produced for each pulse and the interval of time between two pulses are adjusted by adjusting the heat energy supplied by the heating means and by adjusting the high and low thresholds of the valve 9.

In the example of the figures, the valve 9 is incorporated into the handpiece, but it is also possible to provide for it to be fitted to the catheter connected to this handpiece, with a view to achieving the same result.

In the example of the figures, the handpiece 1 further comprises a filling connector 11, advantageously of standardized format, and by means of which the reservoir is filled with the liquid suited to the treatment. As visible in FIG. 2, the handpiece 1 is provided with a nonreturn valve 12 interposed between its reservoir 6 and the filling connector 11 so that the contents of the reservoir 6 cannot reflux toward the container 4 containing the filling liquid.

Another possibility may be to provide filling means comprising an opening fitted with a screw-in stopper for sealing this filling opening of the reservoir in fluidtight manner once filling is over.

This nonreturn valve 12 thus, where appropriate, allows the container 4 to be kept connected to the handpiece 1 during treatment, which allows the reservoir 6 to refill as treatment progresses. It is also possible to position the container 4 at a height, it then being possible for this container to be a pouch containing the liquid which pouch is then hung from a support. In that case, the reservoir can be supplied more or less continuously and naturally by the pouch under the effect of gravity applied to the liquid it contains, so that the reservoir in the handpiece is filled gradually in pace with the emptying thereof.

The handpiece 1 may even be equipped with a purge valve 13 allowing the inside of the reservoir 6 to be connected to the external environment. This purge valve 13 is, for example, actuated by hand in order to expel some of the steam or gas present inside the reservoir 6 during or after the filling thereof.

Aside from the electrically conducting elements, the components of the handpiece 1, such as the reservoir 6 thereof in particular, are made from a heat-resistant plastic, using an injection molding or some other method, so as to have a low production cost. This handpiece 1 may thus advantageously be designed to be disposable after a single use.

In this respect, the handpiece 1 may be produced in different sizes each corresponding to a reservoir volume (capacity), to be chosen according to the dimensions of the vein or of the vessel that is to be treated. The practitioner then chooses a handpiece for which the larger the vein to be treated, the higher the capacity of the reservoir.

The handpiece 1 is electrically powered by the control unit 3 which delivers to the heating resistance 7 an electrical power that allows the liquid contained in the reservoir 6 to be vaporized. The amount of heat introduced into the handpiece 1 may thus be estimated and regulated directly from the unit 3 by monitoring within this unit the electrical power that is actually consumed in the handpiece 1.

It is thus possible to avoid too great an increase in the temperature in the reservoir simply by controlling the voltage and/or the intensity of the current supplied by the unit 3 to the handpiece 1. The unit 3 can thus be used to limit the electrical power injected into the handpiece 1 in order to prevent the temperature in the reservoir 6 from becoming too high. It is found in practice that exchanges of heat between the heating element and the steam are not as good as exchanges of heat between the heating element and the liquid. This results in a risk of overheating in the reservoir, which risk increases as the quantity of liquid in the reservoir decreases.

In order to improve the control of the temperature and state of the mixture in the reservoir 6, this reservoir is advantageously fitted with a thermal probe 14 which measures temperature and which is connected to the unit 3. The intensity of the current (or the voltage or power) supplied to the handpiece 1 can thus be limited by a maximum reference temperature that must not be exceeded. The unit 3 thus makes it possible to increase the intensity if the temperature is below the reference value and reduce it if not.

For example, the regulating valve 9 is set to two bar in order to release steam when it reaches two bar and therefore one hundred and twenty degrees, and the control unit 3 is configured to regulate the supply of electricity so that the temperature in the reservoir cannot exceed one hundred and twenty five or even one hundred and thirty degrees.

Thus, if, for some reason or another, the steam is not discharged correctly by the catheter, the temperature of the handpiece cannot increase inconsiderably, and the steam production parameters (temperature and pressure) are not significantly disrupted.

Conversely, at the start of the heating of the liquid, which is then at ambient temperature, the control unit may then significantly increase the electrical power as long as the nominal temperature has not been reached, so as to speed up the start of heating and cause the liquid to start to vaporize more rapidly.

In general, the unit 3 is designed to be connected to an electrical supply network and to the handpiece 1 in order to power it electrically with low voltage, for example with twelve volts direct current, and with an electrical power of the order of one hundred and fifty watts.

The unit 3 is advantageously provided with an interface with a screen and with various control elements that allow the practitioner to limit the maximum temperature, to view the effective temperature in the reservoir and the electrical power consumed in the handpiece 1.

As will have been appreciated, the handpiece 1 is equipped with an electrical connector by means of which it is electrically connected to the control unit 3 in order, on the one hand to receive the current that powers its electrical resistance and, on the other hand, transfer the temperature values measured by the thermal probe 14 to the unit 3.

As far as the standardized connector that accepts the catheter is concerned, this is advantageously a connector of the Luer-Lock type, which corresponds to a standard very commonly used in medical circles, and thereby allows connection to multiple ranges of catheter. The same may even be true of the connector used for filling the reservoir 6, when there is one, which is then likewise advantageously of the Luer-Lock type.

The use of the system according to the invention by a practitioner goes as follows. First of all, the practitioner chooses the catheter 2 to be used, bearing in mind the disorder to be treated, and connects it to the handpiece, after which he fills the reservoir 6 with the liquid and, using the unit 3, chooses the energy to be delivered in order to treat the vein.

In more concrete terms, the unit 3 is designed to display, for example in real time, the amount of heat that has been transferred by the steam carried by the catheter to the treated vessel. This calculation is for example made on the basis of graphs stored in the unit 3 as a result of a campaign of trials, making it possible to determine for various values of electrical power consumed by the heating means, how much thermal power has actually been transferred to a vessel during the course of treatment.

The practitioner can then introduce the catheter 2 into the vessel that is to be treated. Once the catheter is in place, he activates the unit 3 to begin the heating and vaporizing of the liquid contained in the reservoir 6. The heating then takes place until a predetermined nominal temperature for example of one hundred and twenty degrees is reached, it being possible for this temperature, as indicated above, to be governed only by the setting of the outlet valve 9 that regulates the pressure in the reservoir.

When a sufficient time has elapsed, the liquid begins to vaporize in the reservoir 6 until it reaches the pressure high enough to open the regulating valve 9. This opening then brings about the start of diffusion of steam toward the catheter, and therefore toward the vessel being treated.

The practitioner continues to keep the catheter in place and may potentially move it along the vessel in order to distribute the application of heat all along the portion that is to be treated.

Once a sufficient quantity of heat has been applied, the practitioner may withdraw the catheter and consider the treatment to be over. If the capacity of the reservoir is smaller than the volume of liquid to be vaporized for the quantity of heat that needs to be applied, the practitioner may then refill the reservoir in order to continue with the treatment until the sufficient quantity of heat has been applied.

In the usage example above, it is the practitioner who fills the handpiece 1 with the volume of liquid suited to the treatment that is to be performed. However, the handpiece 1 may be a disposable single-use handpiece pre-filled with liquid and available in various sizes each corresponding to a volumetric capacity of the reservoir it comprises. All the practitioner need then do is to select the handpiece 1 that has the size suited to the treatment that is to be carried out (which means to say that has the appropriate volume of liquid). The practitioner then uses the handpiece 1 he has chosen to carry out the treatment, and disposes of it once the treatment is over.

1-9. (canceled)

10. A handpiece for injecting steam into a human or animal vessel in order to heat the vessel, the handpiece comprising: a steam generator for generating steam from a liquid and a connection member for connecting to one of a catheter or a needle that is to be introduced into the vessel in order to inject the steam into the vessel, wherein the steam generator comprises:
a reservoir to contain the liquid, the reservoir being equipped with electrical heating means for heating the liquid in order to vaporize it and produce a stream of steam that diffuses via the connection member, and
a first nonreturn outlet valve interposed between the reservoir and the connection member for preventing fluid from passing from the connection member toward the reservoir, said first nonreturn outlet valve being a pressure-regulating valve which opens to allow steam to pass from the reservoir toward the connector when the pressure in the reservoir has reached a threshold value.

11. The handpiece as claimed in claim 10, comprising means for filling the reservoir including a second nonreturn valve ensuring that liquid and/or steam cannot leave the reservoir via the filling means.

12. The handpiece as claimed in claim 10, in which the heating means comprises an electrical resistance situated inside the reservoir so as to be surrounded by the liquid and/or the steam that is to be heated.

13. The handpiece as claimed in claim 10, in which the reservoir is at least partially pre-filled with liquid that is to be vaporized.

14. The handpiece as claimed in claim 10, further comprising a purge valve for purging the reservoir.

15. The handpiece as claimed in claim 10, in which the first nonreturn outlet valve is a two-threshold pressure-regulating valve:
the first nonreturn outlet valve opening to allow the steam to pass when the pressure in the reservoir reaches a high threshold, and
the first nonreturn outlet valve closing to prevent the steam from passing when the pressure in the reservoir drops below a low threshold lower than the high threshold.

16. A system comprising a handpiece as claimed in claim 10 and a control unit configured to be connected to the handpiece to supply heating means with electricity, the control unit comprising means for limiting the power of the electrical supply to the heating means.

17. The system as claimed in claim 16, in which the handpiece further comprises a thermal probe situated inside the reservoir, and in which the control unit comprises servo control means for controlling the power of the electrical supply to the heating means on the basis of the temperature delivered by the thermal probe and/or on the basis of the temperature of the heating means so as to servo control the electrical power supplied to the heating means to a maximum permissible temperature in the reservoir.

18. A system comprising a handpiece as claimed in claim 16 and a catheter, wherein the catheter is provided with a valve that prevents fluid from passing from the catheter toward the reservoir, in the form of a valve of the nonreturn type and/or of a valve that opens when the pressure in the reservoir reaches a threshold value.

19. The handpiece as claimed in claim 10, comprising wherein the liquid is one of water or physiological liquid.
